(19) United States
(12) Patent Application Publication
(10) Pub. No.: US 2026/0232902 A1
(43) Pub. Date: Aug. 13, 2026

(54) SCANNING ERROR REDUCTION SYSTEM

(71) Applicant: CAREFUSION 303, INC., San Diego, CA (US)

(72) Inventors: Christopher DiLeo, San Diego, CA (US); Michael K. Workman, Carlsbad, CA (US)

(21) Appl. No.: 19/053,234

(22) Filed: Feb. 13, 2025

(51) Int. Cl. A61M 5/172

(52) U.S. Cl. CPC A61M 5/172

(57) ABSTRACT

A scanning error reduction system and method for preventing scanning errors associated with configuring an infusion device is disclosed. A first scancode is rendered on a first display of the first infusion module of the infusion device. Responsive to determining that the indicated therapy is to be provided by a second infusion module coupled to the device, the rendered first scancode is automatically removed from the first display of the first infusion module and, simultaneously, a second scancode associated with the second infusion module is automatically displayed on a second display of the second infusion module, and a user is prompted to scan the second scancode.

### BACKGROUND

This application relates generally to ensuring that an infusion device is properly programmed.

Modern hospital infusion devices include multiple pump channels for infusing multiple fluids simultaneously. These patient care units (“PCUs”) are mobile and able to administer medications to a patient as the patient moves between care areas throughout a medical environment such a hospital organization. During setup of an infusion therapy, a barcode attached a respective channel may be scanned to associate the channel with a medication order. However, scanning barcodes to identify infusion devices or their connected modules have become problematic, particularly when the barcodes are on the back of the device or have faded over time. Moreover, while the barcode for the respective channel may be scanned, the PCU may be used to enter parameters for the therapy. In some instances, the user may inadvertently scan a barcode of an adjacent channel, or an incorrect barcode may have been applied to the channel, thereby causing a disconnect between the therapy being programmed and the identified channel. Such discrepancies can cause undue delay in initiating a critical therapy or worse, the programming of the therapy using misaligned parameters.

### SUMMARY

The subject technology provides an automated infusion module identification system that correctly identifies the infusion channel to be programmed for a given therapy and actively prevents the misidentification of incorrect channels. Unlike legacy systems and processes, the subject technology coordinates presentation of scancodes on the displays of infusion pump modules in a multi-module infusion device—so that a single scancode is displayed for programming a given therapy, while removing scancodes displayed on modules that do not correspond to the therapy. The scancodes can be an optical code such as a barcode, quick response (QR) code, AprilTag, Aztect, Data Matrix, or MaxiCode or the like. In this regard, the PCU gathers identifiers from attached modules upon their attachment, encodes the identifiers into a recognized symbology, and then displays the scancode at the appropriate time as user interactions are to occur with the given infusion module.

According to various implementations, the subject technology includes a patient care unit, comprising a plurality of infusion modules comprising a first infusion module and a second infusion module, each infusion module comprising an infusion pump configured to pump a fluid according to a respective therapy associated with a patient, wherein the patient care unit is configured to: render, on a first display of the first infusion module, a first scancode associated with the first infusion module; receive, by the patient care unit, an indication of a therapy to be provided; determine that the indicated therapy is to be provided by the second infusion module; responsive to determining that the indicated therapy is to be provided by the second infusion module: remove the rendered first scancode from the first display of the first infusion module; and prompt a user to scan a second scancode associated with the second infusion module, on a second display of the second infusion module. Other aspects include corresponding devices, methods, systems and computer program products for implementation of the corresponding patient care unit and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts an example of an institutional patient care system of a healthcare organization, according to aspects of the subject technology.

FIG. 1B is another view of a portion of the example patient care unit shown in FIG. 1A, according to aspects of the subject technology.

FIG. 2 depicts an example scancode presented by a display device associated with a functional module/channel of a patient care unit, according to aspects of the subject technology.

FIG. 3A depicts an example system for generating and displaying a scancode in a display of a patient care unit, according to aspects of the subject technology.

FIG. 3B depicts an example workflow for preventing scanning errors associated with configuring an infusion device, according to aspects of the subject technology.

FIG. 4 depicts a first example process flow diagram for preventing scanning errors associated with configuring an infusion device, according to aspects of the subject technology.

FIG. 5 depicts a second example process flow diagram for preventing scanning errors associated with configuring an infusion device, according to aspects of the subject technology.

FIG. 6 is a conceptual diagram illustrating an example electronic system for preventing scanning errors associated with configuring an infusion device, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Instead of adding a physical barcode or other identifier to an infusion device for the purpose of scanning or manually entering it later into an external system to identify the device to that system, the infusion device can, when activated, present a scancode on a display screen. The scancode may be presented on an infusion module without having the user being logged into the module and/or associated PCU. In some implementations, a module may be instructed (e.g., by the PCU) to display a scancode on receiving a selection of a therapy to be provided to patient. For example, when a therapy is initiated, the clinician may indicate the module/channel to administer the therapy. On indication (e.g., at the PCU), the module responsible for administering the therapy may be instructed to present a scancode.

According to various implementations, a first scancode may have already been presented on an infusion module connected to a PCU when a therapy is selected to be administered by a different infusion module connected to the PCU. The subject technology may remove the first scancode responsive to the therapy being selected, and then present a second scancode on the infusion module which is to administer the therapy. In this manner, the clinician user is prevented from using the first scancode in connection with the therapy and forced to use the scancode that correctly corresponds to the therapy and the module designated to administer the therapy.

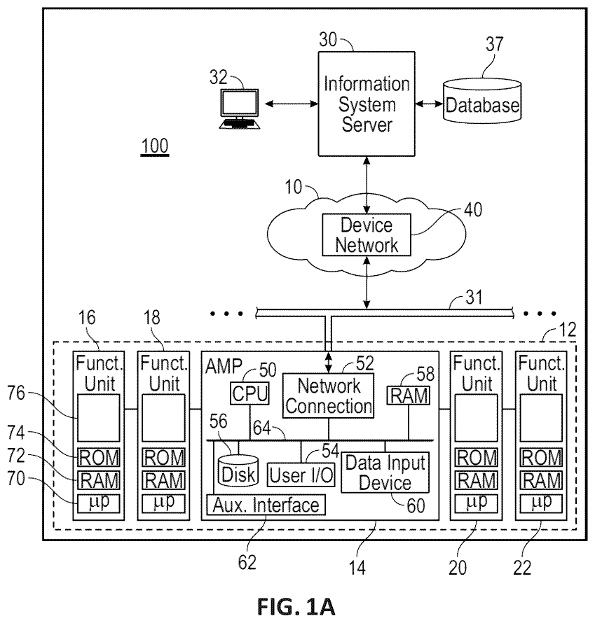

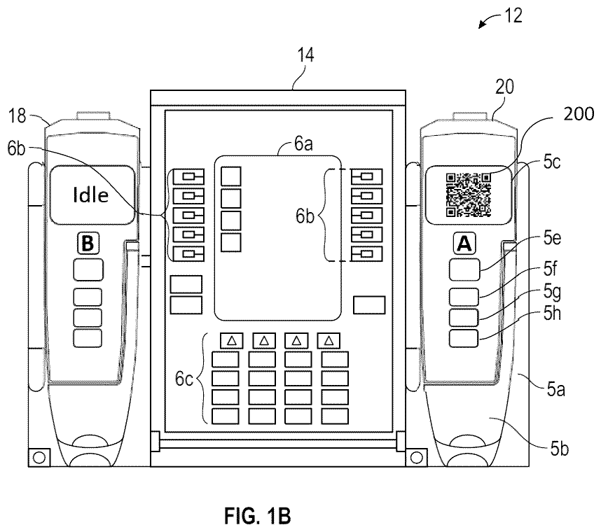

FIG. 1A depicts an example of an institutional patient care system 100 of a healthcare organization, according to aspects of the subject technology. In FIG. 1A, a patient care unit (or “medical device” generally) 12 is connected to a hospital network 10. The term patient care unit (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a network transmission channel 31. Network transmission channel 31 is any wired or wireless transmission channel, for example an 802.11 wireless local area network (LAN). In some implementations, network 10 also includes computer systems located in various departments throughout a hospital. For example, network 10 of FIG. 1A optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care units 12 (and other devices) communicate in accordance with normal operations.

Additionally, institutional patient care system 100 may incorporate a separate information system server 30. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, and mobile devices such as laptops, tablet computers, augmented reality devices, or smartphones, configured with software for communications with information system server 30 via network 10.

Patient care unit 12 comprises a system for providing patient care. Patient care unit 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care unit 12 comprises a control module 14 connected to one or more functional modules 16, 18, 20, 22. Control unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Control unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen (e.g., 6a of FIG. 1B). Additionally, or in the alternative, user interface device 54 could include any means for displaying and inputting information, such as a monitor, a printer, a keyboard, softkeys, a mouse, a track ball and/or a light pen. Data input device 60 may be a code reader capable of scanning and interpreting data printed in a coded format (e.g., a scancode). Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown as disposed within control unit 14, it is recognized that data input device 60 may be associated with a pharmacy system or located externally and communicating with pharmacy system through an appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 1B, at least one of functional modules 16, 18, 20, 22 may be an infusion pump module such as an intravenous infusion pump for delivering medication or other fluid to a patient. Each of functional modules 16, 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 16, 18, 20 and/or 22 may also included a printer, scanner, bar code reader, near-field communication reader, RFID reader, or any other peripheral input, output or input/output device.

Each functional module 16, 18, 20 and/or 22 communicates directly or indirectly with control unit 14, with control unit 14 providing overall monitoring and control of device 12. Functional modules 16, 18, 20 and/or 22 may be connected physically and electronically in serial fashion to one or both ends of control unit 14 as shown in FIG. 1B. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care unit 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1A, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 16.

While each functional module may be capable of a least some level of independent operation, control unit 14 monitors and controls overall operation of device 12. For example, as will be described in more detail below, control unit 14 provides programming instructions to the functional modules 16, 18, 20, 22 and monitors the status of each module.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care unit 12 and network 10 may communicate via automated interaction, manual interaction or a combination of both automated and manual interaction. Automated interaction may be continuous or intermittent and may occur through direct network connection 52, or through RS232 links, MIB systems, RF links such as BLUETOOTH, IR links, WLANS, digital cable systems, telephone modems or other wired or wireless communication means. Manual interaction between patient care unit 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in an information system server 30 (e.g., health information system (HIS) server, decision support, or remote data server), hospital department or unit stations 32, or within patient care unit 12 itself.

All direct communications with medical devices operating on a network in accordance with the subject technology may be performed through server 30. In accordance with aspects of the subject technology, network interface modules incorporated into medical devices such as, for example, infusion pumps or vital signs measurement devices, ignore all network traffic that does not originate from an authenticated RDS. In some implementations, server 30 may coordinate tracking of the location and status of all networked medical devices that have NIMs, and maintain open communication.

According to various implementations, server 30 may include a formulary and/or pharmacy information system. Pharmacy information systems may enable a safer physician medication order process. A pharmacy website (e.g., provided by the server) may provide the physician with a list of available drugs from which the physician may select (stored, e.g., in a database 37). The pharmacy website may contain a drug library having the list of available drugs but may also contain and present to the physician the drug names associated with recommended dosages and dose limits that have been established or adopted by the healthcare facility. In such a case where the physician need only select items from the computer screen rather than having to manually type in drug names and drug administration numbers (such as infusion rates, times, etc.) associated with administration of the medication, a more accurate medication process should result.

If a clinical order is for administration of a particular medication regimen, the order will be transmitted to the facility's pharmacy information server 30. The pharmacy reviews the order, and once the order has been prepared, the order may be transmitted to the nurse station (e.g., at workstation 32) for matching with the appropriate patient. Formulary is an approved list of drugs for use (e.g., available to order for a patient) within a medical facility. Within a formulary, there may be indication for use information and/or concentrations and drug ranges approved for the facility. As will be described further, a formulary may be used to define one or more medical device drug libraries, which may then be provided to infusion pumps within a hospital network. Inside the library, there is medication information such as drug names, concentration, diluent volume, strength, minimum or maximum infusion parameters for a drug, and other parameters. The formulary's establishment of these parameters, along with parameters for off-formulary orders, via the server 30 is useful for maintaining consistency across the healthcare environment and ensuring an order is intelligible and executed according to expectations by other devices within the system (e.g., an infusion pump).

With further reference to FIGS. 1A and 1B, patient care unit 12 is capable of operating in several different modes, or personalities, with each personality defined by a configuration database. The configuration database may be a database 56 internal to patient care unit, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care unit's 12 location in the hospital or hospital computer network. Patient care information may be entered through interface device 52, 54, 60 or 62, and may originate from anywhere in network 10, such as, for example, from a pharmacy server, admissions server, laboratory server, and the like.

A memory 56, 58 of the control unit 14 may contain a drug library or libraries, an event log or logs, and pump configuration settings, such as, but not limited to, profiles to be used in particular practice areas such as ICU, PED, etc. The memory may be electronically loadable memory such as non-volatile memory (e.g., EEPROM). Drug libraries stored on pumps (which illustratively contain such information as the drug names, ranges of delivery parameter values such as proper concentrations, dosage units, and dose limits) can be used to perform drug calculation-based infusions in a clinical setting.

A drug library stored within the pump's memory may include clinical order settings such as limits set by the clinical institution for each drug of the library (also termed as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward of the medical institution in which the drug is being used (for example neonatal care unit (NCU), the intensive care unit (ICU), etc.), and depending on other factors. An alarm may be provided if the nurse sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The pump also includes a display for displaying a user interface, including a control panel through which the user can program the programmable controller and a display screen for displaying drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers from which to make a selection when the electronically loaded drug library is in the pump. The loaded drug library may include a list of syringe sizes identifying syringes that can be used in the drug infusion pump, and the drug infusion pump offers the user the list of syringe sizes from which to make a selection when the electronically loaded drug library is in said pump. In the case of a peristaltic pump, the electronically loaded drug library may include a list of infusion set manufacturers. A loaded drug library may include a set of features, each of which is either be toggled on or off, and the pump offers the user only the features from among the set of features that are toggled on when the electronically loaded drug library is in said pump.

FIG. 1B is another view of a portion of the example patient care unit 12 shown in FIG. 1A, according to aspects of the subject technology. FIG. 1B shows two of the fluid infusion pumps 18, 20 mounted at either side of a control unit 14, and the displays and control keys of each, with the programming module being capable of programming both infusion pumps. The infusion device includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 20. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 20, such as alert indications (e.g., alarm messages).

Display 5c of each pump module 18, 20 may present a scancode 200 for identifying the module. As will be described further, each scancode may facilitate programming of the pump module (and/or PCU) by way of directly providing configuration information to a scanner based system when the scancode is scanned by a scanner. The system may then process the information and provide programming instructions back to the PCU 12 (or module) via a network transmission channel 31 and network connection 52. Control keys 5e-h exist for programming and controlling operations of the infusion pump as desired including, for example, for activating a presentation of a respective scancode on the display 5c. In some implementations, the control keys may be presented as interactive elements on the display 5c (e.g., touchscreen display). The infusion device and/or infusion pump may also include audio alert equipment in the form of a speaker (not shown).

The control unit 14 of the infusion device 12 includes a display 6a for visually communicating various information, such as the operating parameters of a connected pump and alert indications and alert messages, and control keys 6b and 6c for selecting and/or setting control parameters and/or options for controlling the infusion device 12 and connected modules. The control unit 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The control unit 14 may include one or more communications systems (as described with regard to FIG. 1A) with which the control unit 14 may communicate with external equipment such as a medical facility server or other computer and with a portable processor, such as a handheld communication device or a laptop-type of computer, or other information device that a clinician may have to transfer information as well as to download drug libraries to a programming module 16, 18, 20, 22 (such as pump 20). A communication module may be used to transfer access and interaction information for clinicians encountering the programming module or device coupled therewith (e.g., pump 20 or bar code scanner). As described previously, the communications system may include one or more of a radio frequency (RF) system, an optical system such as infrared, a BLUETOOTH™ system, or other wired or wireless system. The bar code scanner and communications system may alternatively be included integrally with the infusion pump 20, such as in cases where a programming module is not used, or in addition to one with the control unit 14. Further, information input devices need not be hard-wired to medical instruments, information may be transferred through a wireless connection as well. Additionally, other types of modules may be connected to the pump modules or to the programming module such as a syringe pump module, patient controlled analgesic module, End Tidal CO₂ monitoring module, oximeter monitoring module, or the like.

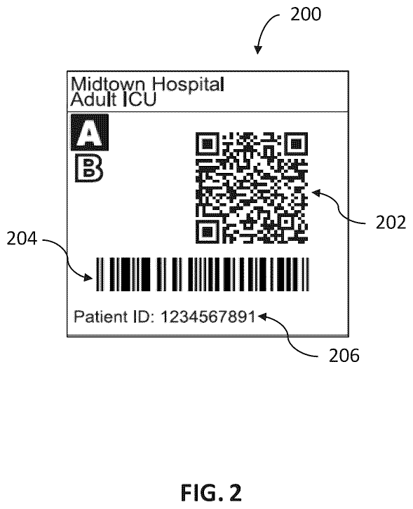

FIG. 2 depicts an example scancode 200 presented by a display device associated with a functional module/channel of a patient care unit (PCU) 12, according to aspects of the subject technology. The subject technology provides each scancode for display as a visualization (e.g., a graphical representation displayed on a display screen). The visualization may include, for example, a linear or two-dimensional (and in some implementations three-dimensional) pattern 202 that may be scanned and recognized and/or converted to digital information. Different devices may receive different visualizations. For example, a first device may receive a traditional barcode visualization (which can, e.g., include multiple codes displayed linearly), while a second device may receive a QR code visualization having a 2D digital pattern(s) of information along an x and y axis. Both visualizations may include the particular arrangement of information of the respective information generated for a particular infusion channel and/or therapy being provided by the infusion channel.

The term “scancode” may be used herein in the singular to represent a single code or identifier, or may include multiple code or identifier representations 202, 204, 206 as depicted in the example of FIG. 2. In some implementations, the plural “scancodes” may be used to describe multiple code or identifier representations (e.g., FIG. 2 may be construed as displaying multiple scancodes). Also, while the subject technology is described herein with reference to a PCU 12 for an infusion device, the device may include other devices such as dispensing cabinets and devices, or may be modules connected to a PCU 14.

With reference to FIG. 1B, presentation of a scancode 200 may be initiated by activation of a designated control key 5e-h or via touch activation of the display 5c. In some implementations, a module 16, 18, 20, 22 may be instructed to present a scancode by the PCU (e.g., during programming of an infusion therapy). The control may be a button on the module (e.g., 5e-h) or the PCU (e.g., 6b, 6c), or virtual button on a display 5c of the module or a display 6a of the control unit 14. In this regard, a user may select a channel/module but have the symbology displayed on the control unit 14 in the programming workflow of that channel/module. In some implementations, the symbology may be displayed on a respective channel/module when a key press on the control unit 14, or on a channel selection for just the intended channel/module.

In some implementations, the user may activate a control to cause a display of the scancode to toggle between different formats. Accordingly, the user can repeatedly activate the control to toggle the displayed scancode (or its identifiers) between a barcode, quick response (QR) code, AprilTag, Aztect, Data Matrix, or MaxiCode and the like. In some implementations, the available identifiers may be configured as an encoded string with each identifier as a substring element (e.g., prefix and suffix).

When the PCU 12 comes online or is powered on for the first time, control unit 14 may interrogate its ports to determine what modules are attached and active. The PCU gathers multiple fields pertaining to the module and encodes the information into a scancode to be presented on a display of the module(s). Encoded fields may include, for example, one or more of a serial number of the device, an interop (or asset) identifier, a set of digits for a location identifier (e.g., a zip code, care area identifier, or coordinate location), and an address of the device (e.g., an APR address). In some implementations, further information may include a date information pertaining to when the device came online or service date of the device. Each field may be represented by an embedding in the scancode. In some implementations, each field my be represented by one or more characters and/or numerical values, or may take on other representations, including symbols. In some implementations, a scancode may include an identifier for a care area or other location within a particular medical organization. In some implementations, each scancode may be assigned to the care area or location (e.g., by way of including the location identifier), and provided to or updated on the PCU and/or module depending on the current location of the device.

In some implementations, the PCU 12 or module devices may directly interact with a server 30 to request and receive the scancode or instructions for generating all or a portion of the scancode 200 from the server. In some implementations, the PCU and/or module may provide its information to the server and the server may obtain the required information, create the scancode, and relay a visualization of the scancode back to the device. In some implementations, the device may forward its serial number and the serial numbers of its attached module(s) and current location (which the server 30 may use to index a database 37 for information). Additionally or in the alternative, the device may make an API (application programming interface) call to the server, indicating that the device requires a scancode for its current location, filled in with the information specific to the area and/or the scanning software used to scan the device within the area (e.g., for asset tracking purposes). The server coordinates the information, and a visualization of the template is provided back to the device for display on a display screen 6a or 5c of the device. A user may then scan the visualization of the template directly from the display.

In some implementations, each module may display a scancode pertaining to an active infusion being administered by the module. In this regard, if a drug is running on both modules 18, 20 of FIG. 1B (A and B), each channel may display a scancode 200 that includes information to identify the module as well as the therapy being administered by the module. In some implementations, when a therapy is activated or programmed into the PCU, the system (control unit 14 or server 30) may obtain information about the therapy and update the respective module's scancode with information pertaining to the therapy. For example, as depicted in FIG. 2, the displayed scancode 200 may include a code 202 representative of the module (including address information) and a code 204 representative of a medication provided by the therapy. In some implementations, the displayed scancode 200 may include a patient identifier 206. In some implementations, a medication identifier 204 and a patient identifier 206 may be omitted as separate codes and instead integrated into a single code 202 together with the module identifier and other pertinent information.

In one example, when multiple scancodes 200 are displayed on multiple infusion modules, a clinician may scan a scancode of a module to direct a change to a therapy (e.g., via an automated programming request). If the clinician inadvertently scans a scancode for module A to make changes to a therapy being administered by module B, when the PCU receives the command the PCU may detect a misalignment between the programming information sent to the module and the current therapy being administered by the module. Rather than generating an error, the PCU may determine that the parameters of the instruction (e.g., medication identifier) aligns with the therapy programmed on module B. In response, the PCU may automatically disable (e.g., remove) the scancode on module A and instruct module B to present a scancode (if not already displayed), while alerting the clinician user (e.g., on the PCU or module A or B) that the wrong scancode was scanned and that the clinician user should rescan the scancode displayed on module B. Accordingly, the clinician user immediately understands which scancode should be scanned to effectuate programming of the selected therapy.

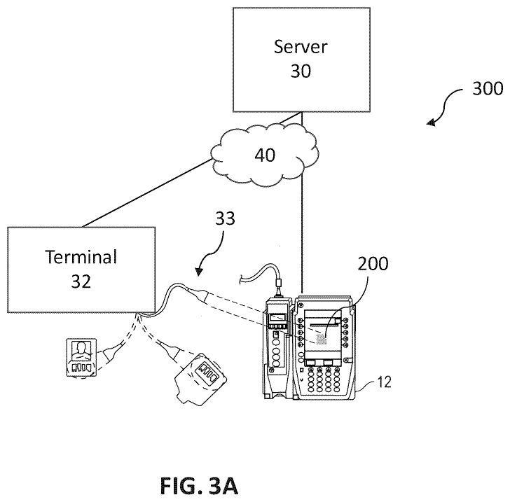

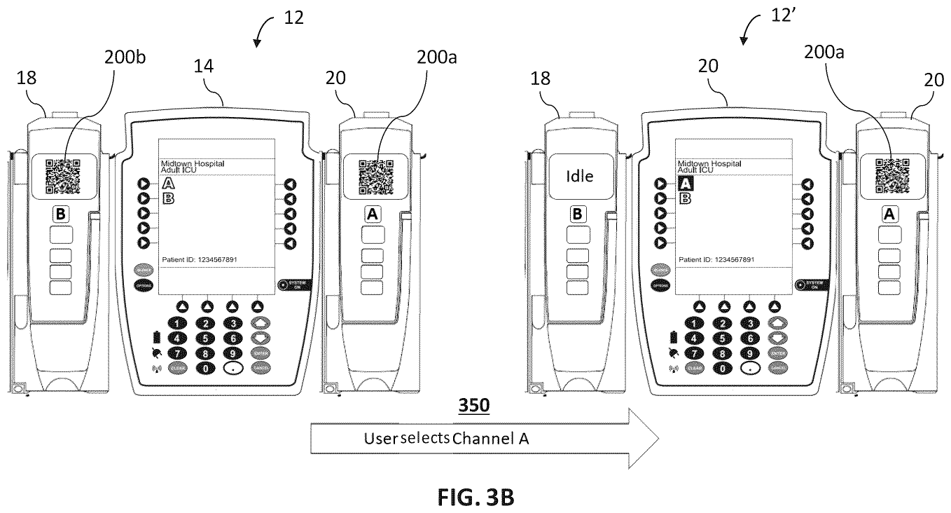

FIG. 3A depicts an example system 300 for generating and displaying a scancode in a display of a patient care unit (PCU) 12, according to aspects of the subject technology. In the depicted example, the scancode 200 is displayed as a graphical visualization on a display screen 6a of the control unit 14 of patient care unit 12. However, it is understood that the graphic visualization may be displayed directly on the designated module (e.g., a connected module 18, 20), as previously described. Also as described previously, the displayed scancode 200 may include a graphical depiction of one or more identifiers.

According to various implementations, the presentation of the scancode 200 may result from activation of a control (e.g., a button or virtual button) at the PCU. In some implementations, the PCU may currently display status information and the activation of the control causes the status information that is currently displayed on the module to be removed and replaced with the presentation of the scancode 200.

In the clinical field, a clinician user may use a scanner 33 (e.g., an EMR system scanner) to scan a scancode 200 to facilitate configuring of the PCU and/or module, or in conjunction with an administration of a therapy. The scanner 33 may be an electronic code reader (optical, RFID, or other data input device) used to scan the scancode 200 presented on a display of the control unit or module. The reader/scanner 33 is not required to be integrated with the medical device 12 but, rather, may be part of a separate device such as a medical records terminal 32 (e.g., part of one or more computing devices) connected to the same network 40 as the PCU 12.

In some implementations, the scanning may be related to an inventory process for maintaining and/or tracking assets of a hospital organization, or related to programming the device. For example, the scanned information may be used to store and/or associate a scanned identifier in the scancode with asset tracking information, or to look up asset information based on the scanned identifier.

In some implementations, the scanning may be for associating an infusion module with another item or device such as a medication to be administered by the infusion module, or with a patient designated to receive the medication or to whom the infusion module is to provide an infusion of the medication. In some implementations, a medication identifier may be scanned to obtain information about the medication prior to programming the infusion device. In some implementations, a patient identifier may be scanned to obtained information about the patient.

In some implementations, the patient identifier, medication identifier, and an order identifier may all be scanned to validate the therapy by ensuring that the right medication is being administered to the right patient at the right time. As described previously, a single scancode 200 may include (or encode) all three identifiers 202, 204, 206 so that the may each be consecutively scanned from the same location on the device. In some implementations, all of the identifiers may be combined in a single code 202, and the scanning of the scancode 200 (including code 202) may allow the system to receive all of the identifiers at once for display to the clinician (on the device display) and/or to associate the identifiers and/or validate the therapy with a single scan.

According to various implementations, the display of the scancode 200 may be initiated, for example, when an infusion channel (module) is indicated for delivery of a given therapy. The clinician user may activate a control 6b or 6c on the medical device to indicate (or select) the channel and the scancode 200 corresponding to the indicated channel may automatically be displayed on a display of the module corresponding to the channel. In some implementations, the user may activate a control 5e-h on a medical device module 18, 20 and interact with and/or scan an identifier displayed on display 5c.

In some implementations, a scanning of a scancode 200 may initiate a process by which information embedded within the scancode (e.g., the device or patient and/or medication identifier(s)) is scanned by the scanner 33 and automatically sent to a centralized server 30 via a network 40. For example, the scancode 200 may be displayed on a module may be used to identify the module (and the patient and a medication) to an EMR (electronic medical records) system, and to send information to the EMR to initiate an automated programming request (APR) to the PCU 12.

In some implementations, with brief reference to FIG. 2, a scancode 200 may include a number of identifiers 202, 204, 206 corresponding to how much information has been programmed into the infusion pump 12. For example, if the medication has been programmed then the scancode may include the medication (in addition to the device identifier). If a patient has been assigned to the infusion device then the scancode may also include an identification of the patient, and so on. In this regard, a user may start an order manually and then scan the scancode with an EMR scanner to upload the information (e.g., device, drug, and patient) to the EMR server to complete the order on the EMR server for the purposes of an APR. As described previously, in some implementations, a single scancode 200 may include or encode multiple identifiers so that the information programmed into the device may be uploaded to the EMR by way of a single scan (e.g., code 202 may include codes 204, 206).

Additionally or in the alternative, the infusion module may include NFC or other short range broadcasting technology that can allow scanning of broadcast information during the same time window as the scancode is shown on the module. An NFC (or RFID) chip may update identifiers based on the identifiers available in the same manner as the corresponding scancode, and provides the available identifier for scanning by RFID/NFC scanner.

An APR may be initiated to load parameters pertaining to an order that was generated or completed on an EMR server, over a network connection, to configure the designated module to administer a therapy. In this regard, the scanning initiates a process by which information pertaining to the item (e.g., scanned from a code affixed to or transmitted by the item) is automatically sent to the hospital's EMR (e.g., at a centralized server 30) via a network 40. The EMR may confirm the information and generate and send the APR to the PCU 12 to load parameters pertaining to the item in a memory of the control unit 14 for the purpose of programming the respective module responsible for the therapy. In some implementations, the APR activates a drug library stored on the device, and the infusion device is programmed according to parameters stored in the drug library for the medication identified in the APR.

In some implementations, a channel may automatically be indicated for programming based on information provided to the PCU 12. For example, the PCU may display several infusion therapies or orders programmed into the device and, on selecting one of the therapies or orders, the PCU may identify the channel that is currently designated by the order and indicate the module/channel for display of a scancode. As will be described further, any other displayed scancodes may then be automatically removed from the display(s) of other module(s) to prevent the erroneous scanning of a scancode associated with a different therapy.

In one example, an APR may be sent down to the PCU 12 to program a particular module. When the APR is received by an infusion device 12, the infusion device 12 attempts to program itself according to the parameters of the APR. If a scancode is not currently displayed by the module that is the subject of the APR then the PCU may instruct the module to automatically present the scancode to identify the module being programmed, and to allow further scanning for updates to the module and/or for other actions related to the scanning described herein. If any other modules are presenting scancodes when the APR is received the other modules may be instructed to remove their scancodes.

In some implementations, upon successful programming of a therapy/order (e.g., manually or by APR), the infusion device may confirm the automatically entered parameters. The confirmation may include presenting one or more user interface screens including the parameters and values along with a control element (e.g., a button) that, when activated, causes the infusion device to begin operation based on the parameters. The user interface may include additional or alternative control elements to allow a clinician to adjust an automatically entered parameter based on, for example, professional judgement or changes in patient condition.

In some implementations, a scancode (or scancodes) may be turned off upon a scanning of the scancode, upon selecting a medication, or in response to a lack of interaction with the pump for a predetermined period of time (e.g., after a time out of 10, 30, 60, 120 seconds from initial presentation). In some implementations, the scancode may be disabled/turned off when the status of the module is changed, for example, from infusing to paused, or vice versa. In some implementations, the scancode may be disabled/turned off when an alarm is generated for the infusion module. For example, if a scancode is being displayed on or for the infusion module corresponding to channel A and then an alert is generated for channel A, the scancode may be automatically removed from the infusion module responsive the alarm. In some implementations, the scancode of one module may be turned off when a different channel is selected via the PCU or module channel select button.

In some implementations, the PCU may detect when more than one module is connected on a single side of the PCU and, when there are two modules that are adjacent (vertically or horizontally) to each other on the same side, the PCU will only render a scancode on one of the adjacent modules. In this manner, multiple scancodes may still be displayed simultaneously but only if on different sides of the PCU (wherein they are substantially separated by the control unit 14). In some implementations, the PCU may render more than one scancode on a single side if the modules are separated by another module, but modules adjacent to each other are prevented from simultaneously displaying scancodes. For example, four infusion modules may be connected to a PCU on a single side. If the first module (nearest the control unit 14) is indicated for programming (e.g., by the user) then the system may cause the scancodes displayed on the second module to be disabled while allowing the third module and/or the fourth module (furthest from the control unit) may remain active.

FIG. 3B depicts an example workflow 350 for preventing scanning errors associated with configuring an infusion device, according to aspects of the subject technology. As will be described further, the subject technology coordinates presentation of scancodes displayed on respective infusion modules so as to reduce the ability to inadvertently scan the code by blocking the scanning of the wrong channel. In this regard, when a channel is indicated for programming, the PCU may prevent all modules other than the module corresponding to the indicated channel from displaying a scancode, while causing the display of the pertinent scancode on the module corresponding to the indicated channel.

For example, each module may initially have a scancode displayed on its respective display by default and, in response to the specific channel being indicated, the PCU may instruct all modules other than the indicated module to remove their scancodes from their displays. For example, as depicted in FIG. 3B, a PCU 12 may currently display a scancode 200a, 200b in each display of two connected infusion modules 18, 20. When the clinician user initiates programming of an order for a therapy on the PCU, the clinician user indicates which channel the therapy will be provided; for example, the user has the choice to select module A or B in the depicted example. Responsive to determining that module A is indicated for therapy, the PCU 12′ causes the scancode 200b pertaining to infusion module B (i.e., module 18) to be removed from the display of infusion module B. The clinician user is then prompted to scan the scancode 200a associated with infusion module A (i.e., module 20), on the display of infusion module A. The prompting may be by way of highlighting the channel corresponding to module A in the display of the PCU (as depicted) or may be by virtue of the scancode 200a being the only remaining scancode. Accordingly, if two or more scancodes are displayed at the same time, the subject technology prevents a user from scanning multiple scancodes at the same time or the wrong scancode (e.g., on an adjacent module).

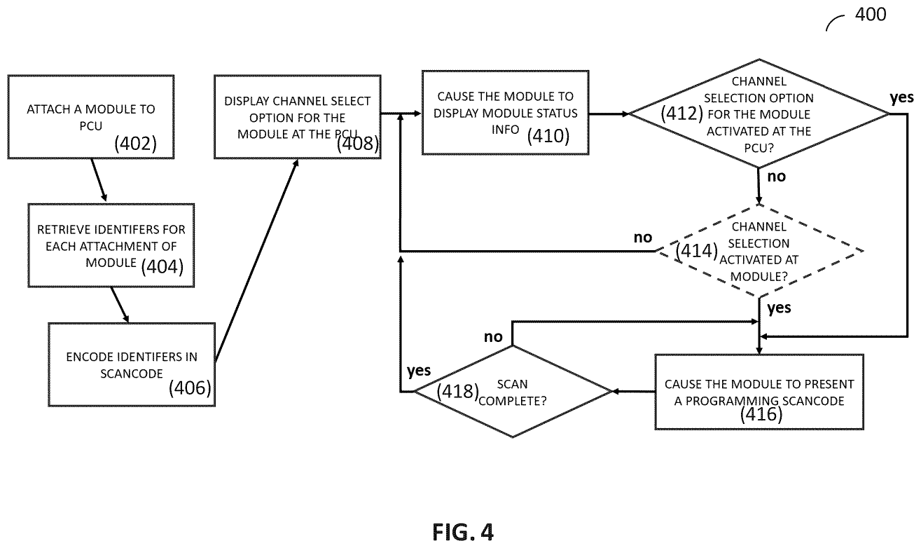

FIG. 4 depicts a first example process flow diagram 400 for preventing scanning errors associated with configuring an infusion device, according to aspects of the subject technology. For explanatory purposes, the various blocks of example process flow 400 are described herein with reference to FIGS. 1-3, and the associated components and/or processes described herein.

In the depicted example, an infusion module 18, 20 is attached (402) to a control unit 14 of a PCU 12. Responsive to each module being attached, the control unit retrieves identifiers for each respective module (404). According to various implementations, the control unit may encode the identifiers in a scancode for the attached module. For example, internally in memory, the control unit may begin to accumulate information for what will be displayed as a scancode for the module. The encoding may be by way of generating and storing the scancode or a portion of the scancode (e.g., a barcode or QR code, or the like) for subsequent display when the channel/module is indicated.

The control unit adds the channel module to the possible user programming selections. In this regard, the channel select option for the module may be displayed at the PCU (408). The process proceeds by the module being caused (e.g., by the control unit 14) to display status information (410) in the module's display.

When the user is ready to program a therapy/order, the user may then select a channel option for the module, to activate the module for the therapy/order. The user may activate the module at the PCU (412) or directly at the module (414). When the module is indicated for the therapy/order, the module is caused to present the previously generated scancode (416), as described previously. The user may then scan the scancode. In some implementations, when scanned by an EMR-associated scanner, the EMR server (e.g., by way of an APR) may initiate programming of the PCU/module over a network, thus indicating that the scan was completed (418). Additionally or in the alternative, the user may indicate the scan as being completed by way of activating a control on the device user interface (e.g., a button on the module or control unit). In some implementations, the scan is determined to be completed by way of a timeout occurring due to lack of further interaction after a predetermined period of time (e.g., 10, 30, 60, 120 seconds from initial presentation of the scancode). In some implementations, the scan is determined to be completed when programming is received for the module, a different module is selected (e.g., via a channel select button), or the status of the module changes (e.g., from infusing to paused, or alarming).

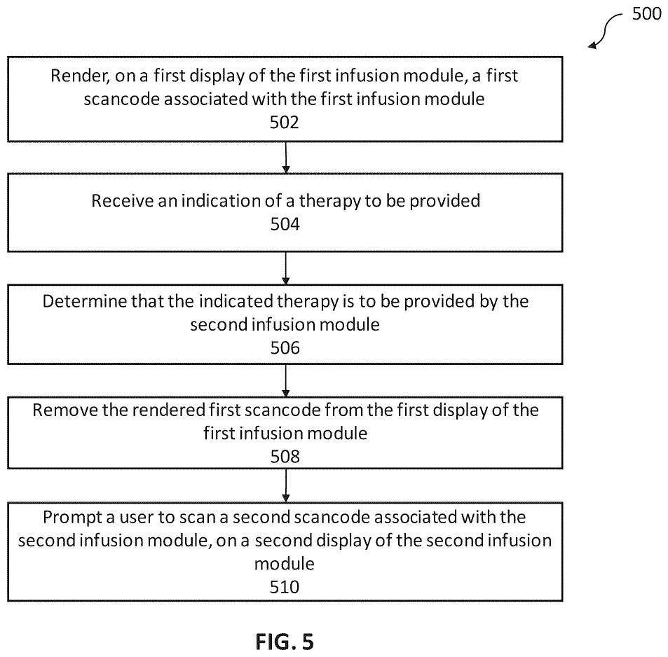

FIG. 5 depicts a second example process flow diagram 500 for preventing scanning errors associated with configuring an infusion device, according to aspects of the subject technology. For explanatory purposes, the various blocks of example process 500 are described herein with reference to FIGS. 1-4, and the associated components and/or processes described herein. The one or more of the blocks of process 500 may be implemented, for example, by a PCU 12 or one or more computing devices associated with the PCU including, for example, server 30, client computing device 32, or associated module 16-20 or control unit 14. In some implementations, one or more of the blocks may be implemented, at least in part, based on one or more machine learning algorithms. In some implementations, one or more of the blocks may be implemented apart from other blocks, and by one or more different processors or devices. Further, for explanatory purposes, to the extent that the blocks of example process 500 are described as occurring in serial, or linearly, in some implementations, multiple blocks of example process 500 may occur in parallel (e.g., blocks 502 and 504 may occur in parallel). In addition, the blocks of example process 500 need not be performed in the order shown and/or one or more of the blocks of example process 500 need not be performed.

As described previously, a PCU may include a plurality of infusion modules—each corresponding to a different infusion channel—connected to (e.g., physically coupled to) a central control unit 14. Each infusion module, when programed appropriately, may be configured to pump a fluid according to a respective therapy associated with a patient.

According to various implementations, the PCU causes a first scancode associated with a first infusion module to be presented on a display of the first infusion module (502). According to some implementations, each connected infusion module may automatically display a scancode by default when connected to the control unit 14. Each scancode may embed one or more identifiers selected from an identifier of the respective module, an identifier of an associated patient, and/or an identifier of medication to be infused by the respective module. In some implementations, the identifiers are embedded in the scancode as the identifiers are made known to the PCU by virtue of a programming of a therapy or order at the PCU (e.g., by way of the user inputting the information, or via a received APR). As described previously, each identifier may be individually recognizable (and thus independently scannable) or may be collectively embedded in a single code.

The PCU receives an indication of a therapy to be provided by the patient care unit (504) and determines that the indicated therapy is to be provided by a second infusion module (506), different than the first infusion module. The indication of the therapy may be received via a user interface of the patient care unit or an automated programming request issued from a server. For example, the therapy may be indicated upon generation of an order at the PCU, or assignment of a medication to an existing order. The PCU may determine which infusion module is indicated for the therapy (e.g., the second infusion module) when, for example, the user selects an infusion channel corresponding to the module for the purpose of programming the therapy. The selection of the channel may occur during programming of the order or during a programmatic update of one or more parameters of an ongoing therapy. In some implementations, the PCU may auto-select the channel (thus indicating the infusion module), automatically, based on a next available channel during programming of the order. In some implementations, the selection of the channel may occur remotely, for example, by APR.

Responsive to determining that the indicated therapy is to be provided by the second infusion module, the PCU automatically instructs the first infusion module to remove the rendered first scancode from the first display of the first infusion module (508) and prompts (510) a user to scan a second scancode associated with the second infusion module, on a second display of the second infusion module. The prompting may include the second scancode being displayed for a first time on the second display. For example, the second scancode may not be displayed on the second display when the therapy is determined to be provided by the second infusion module, but rendered on the second display responsive to determining that the selected therapy is designated by the second infusion module. In some implementations, the prompt includes a display of the second scancode together with a visual or audible alert associated with the module (e.g., on the module or the module's display screen or an indication of the module on a display screen of the PCU).

In some implementations, once displayed, a respective scancode can be toggled between different formats. For example, the user can repeatedly activate the control to toggle the displayed scancode (or its identifiers) between a barcode, quick response (QR) code, AprilTag, Aztect, Data Matrix, or MaxiCode and the like. Additionally or in the alternative, a scancode may include an RFID (radio frequency identifier) of NFC (near field communication) chip that toggles between RFID or NFC formats in the same manner.

According to various implementations, a respective scancode may be scanned to obtain and display information pertaining to an identified item in the scancode. For example, the clinician user may use an EMR-associated scanner or an asset identification scanner to display information about a therapy programmed by the module displaying the scancode, or an item identified by the scancode. For example, if a medication is identified by the scancode, the scanned identifier may be sent to a server, and the server may respond with a display of information about the medication. If a patient is also identified by the scancode then the displayed information may include whether the medication is appropriate for the patient based on known conditions of the patient. The displayed information may include, as pertaining to the module identified in the scancode, information about the order or therapy currently programmed for the module.

Many of the above-described example process 500 and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

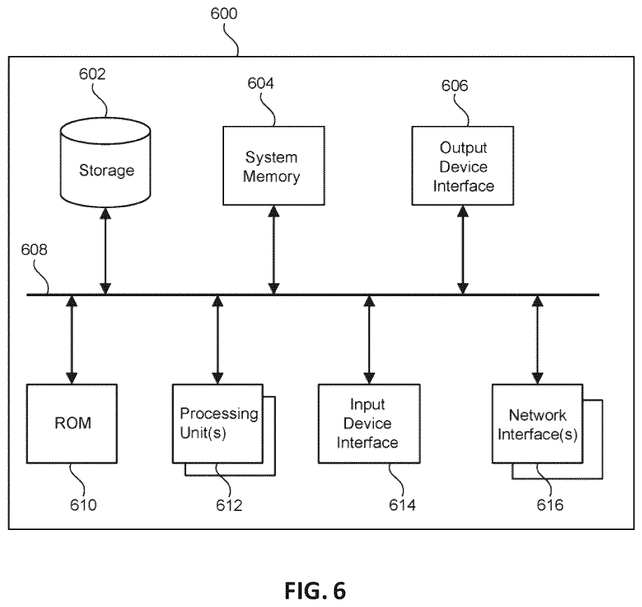

FIG. 6 is a conceptual diagram illustrating an example electronic system 600 for preventing scanning errors associated with configuring an infusion device, according to aspects of the subject technology. Electronic system 600 may be a computing device for execution of software associated with one or more portions or steps of processes 300, 350, 400, and 500 or components and methods provided by FIGS. 1-5, including but not limited to computing hardware within servers 30, 34, terminal 32, 206, medical device 12, and/or any computing devices or associated modules or terminals disclosed herein. Electronic system 600 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and methods previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as, random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations.

Bus 408 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification

Clause 1. A patient care unit, comprising: a plurality of infusion modules comprising a first infusion module and a second infusion module, each infusion module comprising an infusion pump configured to pump a fluid according to a respective therapy associated with a patient; wherein the patient care unit is configured to: render, on a first display of the first infusion module, a first scancode associated with the first infusion module; receive, by the patient care unit, an indication of a therapy to be provided; determine that the indicated therapy is to be provided by the second infusion module; responsive to determining that the indicated therapy is to be provided by the second infusion module: remove the rendered first scancode from the first display of the first infusion module; and prompt a user to scan a second scancode associated with the second infusion module, on a second display of the second infusion module.

Clause 2. The patient care unit of Clause 1, further configured to: determine that the indicated therapy is to be provided by the second infusion module based on a user input for programming the second infusion module to provide the therapy, or based on an automated programming request issued from a server.

Clause 3. The patient care unit of Clause 2, wherein an infusion is currently being provided by the infusion pump of the second infusion module, and indicated therapy comprises a programmatic update of one or more parameters associated with the infusion.

Clause 4. The patient care unit of any one of Clauses 1-3, further configured to: determine that the second scancode was scanned; and responsive to determining that the second scancode was scanned, remove the second scancode from the second display.

Clause 5. The patient care unit of any one of Clauses 1-4, wherein the first scancode is displayed on the first display and the second scancode is displayed on the second display when the indicated therapy is determined to be provided by the second infusion module, and wherein the prompt comprises a visual or audible indication associated with the second infusion module and display of the second scancode.

Clause 6. The patient care unit of any one of Clauses 1-5, wherein prompting the user to scan the second scancode comprises rendering the second scancode on the second display for a first time.

Clause 7. The patient care unit of any one of Clauses 1-6, further configured to: determine that the first infusion module and the second infusion module are physically adjacent to each other, wherein the first scancode is removed from the first display based on the first and second infusion modules being physically adjacent to each other; and while the second scancode is displayed on the second display, restrict input to only parameters associated with the second infusion module providing the indicated therapy.

Clause 8. The patient care unit of any one of Clauses 1-7, wherein a portion of the second scancode displayed on the second display, when scanned, causes a server to instruct the patient care unit to display information pertaining to the therapy, information pertaining to a drug associated with the therapy, or information pertaining to a patient associated with the therapy.

Clause 9. The patient care unit of any one of Clauses 1-8, further comprising: a near field communication device, wherein the patient care unit is configured to wirelessly broadcast, via the near field communication device, information embedded within the second scancode during a same window of time that the second scancode is displayed on the second display of the second infusion module.

Clause 10. The patient care unit of any one of Clauses 1-9, further configured to: provide a control for causing the second display to toggle between a plurality of formats of the second scancode; and in response to receiving an input at the control, toggling from a display of the second scancode in a first format to a display of a second scancode in a second format.

Clause 11. A machine-implemented method for preventing scanning errors associated with configuring an infusion device, comprising: rendering, on a first display of a first infusion module, a first scancode associated with the first infusion module; receiving, by a patient care unit coupled to the first infusion module, an indication of a therapy to be provided; determining that the indicated therapy is to be provided by a second infusion module; responsive to determining that the indicated therapy is to be provided by the second infusion module coupled to the patient care unit: removing the rendered first scancode from the first display of the first infusion module; and prompting a user to scan a second scancode associated with the second infusion module, on a second display of the second infusion module.

Clause 12. The machine-implemented method of Clause 11, further comprising: determining that the indicated therapy is to be provided by the second infusion module based on a user input for programming the second infusion module to provide the therapy, or based on an automated programming request issued from a server.

Clause 13. The machine-implemented method of Clause 11 or Clause 12, further comprising: determining that the second scancode was scanned; and responsive to determining that the second scancode was scanned, removing the second scancode from the second display.

Clause 14. The machine-implemented method of any one of Clauses 11-13, wherein the first scancode is displayed on the first display and the second scancode is displayed on the second display when the indicated therapy is determined to be provided by the second infusion module, and wherein the prompt comprises a visual or audible indication associated with the second infusion module and display of the second scancode.

Clause 15. The patient care unit of any one of Clauses 11-14, wherein prompting the user to scan the second scancode comprises rendering the second scancode on the second display for a first time.

Clause 16. The machine-implemented method of any one of Clauses 11-15, further configured to: determining that the first infusion module and the second infusion module are physically adjacent to each other, wherein the first scancode is removed from the first display based on the first and second infusion modules being physically adjacent to each other; and while the second scancode is displayed on the second display, restricting input to only parameters associated with the second infusion module providing the indicated therapy.

Clause 17. The machine-implemented method of any one of Clauses 11-16, further comprising: broadcasting, via a near field communication device, information embedded within the second scancode during a same window of time that the second scancode is displayed on the second display of the second infusion module.

Clause 18. The machine-implemented method of any one of Clauses 11-17, further configured to: providing a control for causing the second display to toggle between a plurality of formats of the second scancode; and in response to receiving an input at the control, toggling from a display of the second scancode in a first format to a display of a second scancode in a second format.

Clause 19. The machine-implemented method of any one of Clauses 11-18, wherein responsive to determining that the indicated therapy is to be provided by the second infusion module and before removing the rendered first scancode from the first display: displaying an indication that the therapy is to be provided by the second infusion module; and prompting the user, on the second display or on a display of the patient care unit, for confirmation to scan the second scancode on the second display.

Clause 20. A non-transitory machine-readable medium storing instructions thereon that, when executed by a patient care unit, cause the patient care unit to perform a method according to any one of Clauses 11-19:

### Further Consideration:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, . NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A patient care unit, comprising:
a plurality of infusion modules comprising a first infusion module and a second infusion module, each infusion module comprising an infusion pump configured to pump a fluid according to a respective therapy associated with a patient;
wherein the patient care unit is configured to:
render, on a first display of the first infusion module, a first scancode associated with the first infusion module;
receive, by the patient care unit, an indication of a therapy to be provided;
determine that the indicated therapy is to be provided by the second infusion module;
responsive to determining that the indicated therapy is to be provided by the second infusion module:
remove the rendered first scancode from the first display of the first infusion module; and
prompt a user to scan a second scancode associated with the second infusion module, on a second display of the second infusion module.

2. The patient care unit of claim 1, further configured to:
determine that the indicated therapy is to be provided by the second infusion module based on a user input for programming the second infusion module to provide the therapy, or based on an automated programming request issued from a server.

3. The patient care unit of claim 2, wherein an infusion is currently being provided by the infusion pump of the second infusion module, and indicated therapy comprises a programmatic update of one or more parameters associated with the infusion.

4. The patient care unit of claim 1, further configured to:
determine that the second scancode was scanned; and
responsive to determining that the second scancode was scanned, remove the second scancode from the second display.

5. The patient care unit of claim 1, wherein the first scancode is displayed on the first display and the second scancode is displayed on the second display when the indicated therapy is determined to be provided by the second infusion module, and wherein the prompt comprises a visual or audible indication associated with the second infusion module and display of the second scancode.

6. The patient care unit of claim 1, wherein prompting the user to scan the second scancode comprises rendering the second scancode on the second display for a first time.

7. The patient care unit of claim 1, further configured to:
determine that the first infusion module and the second infusion module are physically adjacent to each other, wherein the first scancode is removed from the first display based on the first and second infusion modules being physically adjacent to each other; and
while the second scancode is displayed on the second display, restrict input to only parameters associated with the second infusion module providing the indicated therapy.

8. The patient care unit of claim 1, wherein a portion of the second scancode displayed on the second display, when scanned, causes a server to instruct the patient care unit to display information pertaining to the therapy, information pertaining to a drug associated with the therapy, or information pertaining to a patient associated with the therapy.

9. The patient care unit of claim 1, further comprising:
a near field communication device,
wherein the patient care unit is configured to wirelessly broadcast, via the near field communication device, information embedded within the second scancode during a same window of time that the second scancode is displayed on the second display of the second infusion module.

10. The patient care unit of claim 1, further configured to:
provide a control for causing the second display to toggle between a plurality of formats of the second scancode; and
in response to receiving an input at the control, toggling from a display of the second scancode in a first format to a display of a second scancode in a second format.

11. A machine-implemented method for preventing scanning errors associated with configuring an infusion device, comprising:
rendering, on a first display of a first infusion module, a first scancode associated with the first infusion module;
receiving, by a patient care unit coupled to the first infusion module, an indication of a therapy to be provided;
determining that the indicated therapy is to be provided by a second infusion module;
responsive to determining that the indicated therapy is to be provided by the second infusion module coupled to the patient care unit:
removing the rendered first scancode from the first display of the first infusion module; and
prompting a user to scan a second scancode associated with the second infusion module, on a second display of the second infusion module.

12. The machine-implemented method of claim 11, further comprising:
determining that the indicated therapy is to be provided by the second infusion module based on a user input for programming the second infusion module to provide the therapy, or based on an automated programming request issued from a server.

13. The machine-implemented method of claim 11, further comprising:
determining that the second scancode was scanned; and
responsive to determining that the second scancode was scanned, removing the second scancode from the second display.

14. The machine-implemented method of claim 11, wherein the first scancode is displayed on the first display and the second scancode is displayed on the second display when the indicated therapy is determined to be provided by the second infusion module, and wherein the prompt comprises a visual or audible indication associated with the second infusion module and display of the second scancode.

15. The patient care unit of claim 11, wherein prompting the user to scan the second scancode comprises rendering the second scancode on the second display for a first time.

16. The machine-implemented method of claim 11, further configured to:
determining that the first infusion module and the second infusion module are physically adjacent to each other, wherein the first scancode is removed from the first display based on the first and second infusion modules being physically adjacent to each other; and
while the second scancode is displayed on the second display, restricting input to only parameters associated with the second infusion module providing the indicated therapy.

17. The machine-implemented method of claim 11, further comprising:
broadcasting, via a near field communication device, information embedded within the second scancode during a same window of time that the second scancode is displayed on the second display of the second infusion module.

18. The machine-implemented method of claim 11, further configured to:
providing a control for causing the second display to toggle between a plurality of formats of the second scancode; and
in response to receiving an input at the control, toggling from a display of the second scancode in a first format to a display of a second scancode in a second format.

19. The machine-implemented method of claim 11, wherein responsive to determining that the indicated therapy is to be provided by the second infusion module and before removing the rendered first scancode from the first display:
displaying an indication that the therapy is to be provided by the second infusion module; and
prompting the user, on the second display or on a display of the patient care unit, for confirmation to scan the second scancode on the second display.

20. A non-transitory machine-readable medium storing instructions thereon that, when executed by a patient care unit, cause the patient care unit to perform operations comprising:
rendering, on a first display of a first infusion module coupled to the patient care unit, a first scancode associated with the first infusion module;
receiving, by the patient care unit, an indication of a therapy to be provided;
determining that the indicated therapy is to be provided by a second infusion module;
responsive to determining that the indicated therapy is to be provided by the second infusion module coupled to the patient care unit:
removing the rendered first scancode from the first display of the first infusion module; and
prompting a user to scan a second scancode associated with the second infusion module, on a second display of the second infusion module.
